(19) United States
(12) Patent Application Publication
(10) Pub. No.: US 2014/0234316 A1
(43) Pub. Date: Aug. 21, 2014

(54) VACCIBODIES TARGETED TO CROSS-PRESENTING DENDRITIC CELLS

(71) Applicant: UNIVERSITY OF OSLO, Oslo (NO)

(72) Inventors: Bjarne Bogen, Snaroya (NO); Even Fossum, Oslo (NO); Gunnveig Grodeland, Asker (NO)

(21) Appl. No.: 14/346,182

(22) PCT Filed: Sep. 24, 2012
(86) PCT No.: PCT/IB2012/002330

§ 371 Date: Mar. 20, 2014

Related U.S. Application Data
(60) Provisional application No. 61/538,186, filed on Sep. 23, 2011.

(30) Foreign Application Priority Data
Sep. 24, 2012 (IB) PCT/IB2012/002330

(51) Int. Cl. C07K 14/47; C07K 14/005; C07K 16/18

(52) U.S. Cl. CPC C07K 14/47; C07K 16/18; C07K 14/005
USPC 424/135.1; 424/133.1

(57) ABSTRACT

The present invention relates to recombinant fusion proteins targeted to dendritic cells and uses thereof. In particular, the present invention relates to fusion proteins comprising an antibody component and a targeting components, and uses of such fusion proteins to trigger immune responses.

### CROSS-REFERENCE TO RELATED APPLICATIONS

The present application claims priority to pending U.S. Provisional Patent Application No. 61/538,186, filed Sep. 23, 2012, the contents of which are incorporated by reference in their entireties.

### FIELD OF THE INVENTION

The present invention relates to recombinant fusion proteins targeted to dendritic cells and uses thereof. In particular, the present invention relates to fusion proteins (vaccibodies) comprising a targeting component, an antigen, a linker region and an antibody component and uses of such homodimeric fusion proteins, or DNA encoding such fusion proteins, to trigger immune responses.

### BACKGROUND OF THE INVENTION

DNA vaccination is a technically simple way of inducing immune responses. However, success in small animals has not yet been reproduced in clinical trials. Several strategies are currently being pursued to increase efficacy of DNA vaccines.

Targeting of protein antigens to antigen-presenting cells (APC) can improve T- and B-cell responses. Recombinant immunoglobulin (Ig) molecules are well suited for this purpose. For example, short antigenic epitopes can replace loops between (β-strands in the Ig constant domains while targeted antigen delivery is obtained by equipping the recombinant Ig with variable (V) regions specific for surface molecules on APC. However, such a strategy is unfit for larger antigens containing unidentified epitopes, moreover recombinant Ig molecules with short T cell epitopes fail to elicit antibodies against conformational epitopes. To overcome these limitations, targeted Ig-based homodimeric DNA vaccines (vaccibodies) have been generated that express infectious or tumor antigens with a size of at least 550 aa with maintenance of conformational epitopes.

No DNA vaccine has so far been approved for human use due to lack of efficacy. Also there is no effective vaccine available for several infectious diseases. In particular, no therapeutic DNA cancer vaccine has been approved for human use.

What is needed are DNA vaccines with improved efficacy.

### SUMMARY OF THE INVENTION

The present invention relates to recombinant fusion proteins targeted to dendritic cells and uses thereof. In particular, the present invention relates to fusion proteins (vaccibodies) comprising a targeting component, an antigen, a linker region and an antibody component and uses of such homodimeric fusion proteins, or DNA encoding such fusion proteins, to trigger immune responses.

Accordingly, embodiments of the present invention provides a fusion polypeptide, nucleic acids encoding the polypeptide, as well as vectors and cells comprising the vectors encoding the fusion polypeptide, wherein the fusion polypeptide comprises a targeting unit comprising an amino acid sequence having at least 80% sequence identity to the amino acid sequence of human or mouse Xcl1 or Xcl2 (e.g., as described by SEQ ID NOs 1, 2 or 3) (e.g., at least 85%, 90%, 95%, 99% or 100% homology) and an antigenic unit, the targeting unit and the antigenic unit being connected through a dimerization motif. In some embodiments, the fusion polypeptide preferably binds to Xcr1 on cross-presenting DCs. In some embodiments, variants or homologs of human or mouse Xcl1 or Xcl2 exhibit a higher affinity for Xcr1 than the native human or mouse Xcl1 or Xcl2.

In some embodiments, the antigenic unit is an antigenic scFv, a bacterial antigen, a viral antigen or a cancer associated or a cancer specific antigen. In some embodiments a linker, such as a (G₄S)₃ linker, connects the VH and VL in the antigenic scFv. In some embodiments the antigenic scFv is derived from a monoclonal Ig produced by myeloma or lymphoma cells. In some embodiments the antigenic unit is a telomerase, or a functional part thereof. In some embodiments the telomerase is hTERT. In some embodiments the antigenic unit is a melanoma antigen. In some embodiments the melanoma antigen is tyrosinase, TRP-1, or TRP-2. In some embodiments the antigenic unit is a prostate cancer antigen. In some embodiments the prostate cancer antigen is PSA. In some embodiments the antigenic unit is a cervix cancer antigen. In some embodiments the cervix cancer antigen is selected from the list consisting of E1, E2, E4, E6, E7, L1 and L2. In some embodiments the antigenic unit is derived from a bacterium. In some embodiments the bacterium derived antigenic unit is a tuberculosis antigen. In some embodiments the bacterium derived antigenic unit is a brucellosis antigen. In some embodiments the antigenic unit is derived from a virus. In some embodiments the virus derived antigenic unit is derived from HIV. In some embodiments the HIV derived antigenic unit is derived from gp120 or Gag. In some embodiments the antigenic unit is selected from the list consisting of influenza virus hemagglutinin (HA), nucleoprotein, and M2 antigen; and Herpes simplex 2 antigen glycoprotein D.

In some embodiments, the polypeptide is present as a dimer. In some embodiments the dimerization motif comprises a hinge region and optionally another domain that facilitate dimerization, such as an immunoglobulin domain, optionally connected through a linker. In some embodiments, the dimerization domain comprises human IgG3 dimerization domain (hCH3). In some embodiments the hinge region has the ability to form one, two, or several covalent bonds. In some embodiments the covalent bond is a disulphide bridge. In some embodiments the immunoglobulin domain of the dimerization motif is a carboxyterminal C domain, or a sequence that is substantially homologous to said C domain. In some embodiments the carboxyterminal C domain is derived from IgG. In some embodiments the immunoglobulin domain of the dimerization motif has the ability to homodimerize. In some embodiments the immunoglobulin domain of the dimerization motif has the ability to homodimerize via noncovalent interactions. In some embodiments the noncovalent interactions are hydrophobic interactions. In some embodiments the dimerization domain does not comprise the C_{H}2 domain. In some embodiments the dimerization motif consist of hinge exons h1 and h4 connected through a linker to a C_{H}3 domain of human IgG3. In some embodiments the linker that connects the hinge region and another domain that facilitate dimerization, such as an immunoglobulin domain, is a G₃S2G₃SG linker. In some embodiments the antigenic unit and the dimerization motif is connected through a linker, such as a GLSGL linker. In some embodiments, preferred variant homodimeric proteins have increased affinity for the Xcr1 chemokine receptor as compared to the affinity of the native homodimeric protein.

In some embodiments, the present invention provides nucleic acid molecules encoding the vaccibodies described above. In some embodiments, the nucleic acid molecules according to invention are included in a vector. In some embodiments, the present invention provides host cells comprising the vectors. In some embodiments, the nucleic acid molecule according to the invention is formulated for administration to a patient to induce production of the homodimeric protein in said patient.

In some embodiments the vaccine according to the invention comprises a pharmaceutically acceptable carrier and/or adjuvant. In some embodiments, the present invention provides a vaccine against a cancer or an infectious disease comprising an immunologically effective amount of a homodimeric protein as described above or nucleic acid molecule encoding the monomeric protein which can form the homodimeric protein described above, wherein the vaccine is able to trigger T-cell- and/or B-cell immune response (preferably both) and wherein the homodimeric protein contain an antigenic unit specific for said cancer or infectious disease.

In some embodiments the cancer treated by a vaccine or pharmaceutical compositions according to the present invention is multiple myeloma or lymphoma, malignant melanoma, HPV induced cancers, prostate cancer, breast cancer, lung cancer, ovarian cancer, and/or liver cancer. In some embodiments the infectious disease treated by a vaccine or pharmaceutical compositions according to the present invention is selected from the list consisting of influenza, Herpes, CMV, HPV, HBV, brucellosis, HIV, HSV-2 and tuberculosis.

The present invention further provides kits comprising the fusion polypeptides.

Additional embodiments of the present invention provide a method of inducing an immune response, comprising administering the vaccine compositions described herein to a subject under conditions such that the subject generates an immune response to the antigen unit.

In some embodiments, the present invention further provides methods for preparing a homodimeric protein as described above, the method comprising introducing the nucleic acid molecule encoding the monomeric protein which can form the homodimeric protein described above into a cell population; culturing the cell population; and collecting and purifying the homodimeric protein expressed from the cell population.

Additional embodiments are described herein.

### BRIEF DESCRIPTION OF THE DRAWINGS

FIG. 1 shows the structure and function of Xcl1 targeted vaccibodies. (A) The vaccibody structure with mouse Xcl1 as a targeting unit, a dimerization domain, and a viral antigenic unit. (B) Schematic drawing of targeting using fusion proteins of embodiments of the present invention. Xcl1-vaccibodies bind Xcr1 expressing DC subsets which subsequently present peptides of the viral antigen on MHC-I to CD8+ T-cells, thus inducing a cytotoxic T-cell response capable of killing virally infected cells.

FIG. 2 shows XCR1 expression by DC subsets from nonlymphoid and lymphoid organs. A, Gating strategy defining DC subsets in each organ. B-J, Histograms showing bgal enzymatic activity representative of XCR1 expression in DC subsets of various organs of C57BL/6J and XCR1-bGal mice (except for E where the recombinant XCL1-mCherry was used). B, Epidermis. C, Dermis. D-F, CLNs. F, The percentage of bGal+ cells was calculated by subtracting the percent of bGal+ cells in C57BL/6J mice to the percentage of bGal+ cells in XCR1-bGal mice. G, CADM1 expression in CLN-resident CD11b+ and CD8α+ DCs (left panel) and in CLN-mig CD11b+, CD1032, and CD103+ DCs (right panel). H, Liver, lungs, and intestine. I, Mig-DC subsets from MedLNs and MLNs. J, LT-resident DC subsets from spleen, MedLNs and MLNs.

FIG. 3 shows the characterization of Xcl1 targeted vaccibodies. A) Vaccibodies are expressed in vivo as dimeric proteins consisting of a targeting unit (Xcl1), a dimerization unit (hCH3) and an antigen unit (mCherry). To generate a mutant that putatively did not bind Xcr1 we generated a mutated Xcl1 where cystein 11 was mutated to an alanine (C11A). Xcl1-targeted and the presumptively non-targeting C11A mutant vaccibodies were expressed in 293E cells and analyzed by B) western blotting using antibodies directed against mCherry and C) ELISA with antibodies directed against Xcl1. D) Binding of Xcl1- and Xcl1(C11A)-mCherry vaccibodies to resident CD8α+ DCs (left panel) and CD11b⁺ DCs isolated from spleen. DCs incubated with Xcl1-mCherry, Xcl1(C11A)-mCherry, and DCs not incubated with vaccibodies: E) Lack of binding of Xcl1- and Xcl1(C11A)-mCherry to CD8α+ DCs isolated from Xcr1^{-/-} mice.

FIG. 4 shows humoral immune response to Xcl1 targeted HA-vaccibodies. A comparison was made to HA alone, NIP-HA (a non-targeted vaccibody specific for the hapten NIP) and the C11A mutant. a) Serum samples taken 14 days post immunization were analyzed for anti-HA antibodies. b) The serum immunoglobulin response were further analysed for IgG1 and IgG2a isotype at day 14 post immunization. Serum levels for IgG2a c) and IgG1 d) in Balb/c mice was monitored for 18 weeks, with serum samples being collected on week 1, 3, 5, 7, 10, 14 and 18. e) IgG2a serum response when titrating Xcl1-HA vacciody DNA in Balb/c mice. The numbers in brackets indicate the total amount of DNA used to immunize the mice.

FIG. 5 shows pentamer staining of CD8+ T-cells specific for the HA peptide IYSTVASSL presented on MHC-I molecule K^{d}. a) Cells isolated from draining lymph nodes were gated on CD8 expression (RI) and analysed for binding of the PE-conjugated IYSTVASSL pentamer. The numbers in the top right quadrant indicate the percentage of pentamer positive CD8+ T-cells. b) Summary of the pentamer staining with all the controls included. We observed a significant increase in pentamer positive CD8+t-cells in Xcl1-HA vaccinated mice compared to NIP-HA or C11A-HA vaccinated mice (Mann-Whitney).

FIG. 6 shows that Xcl1-HA vaccibodies protects mice against a lethal challenge of Influenza A/PR/8/34(H1N1) (PR8). a) Mice were challenged with 5× lethal dose of PR8 14 days post immunization and monitored for weight loss after immunization with Xcl1-HA, C11A-HA, HA or NaCl. The experiment was terminated on day 7. b) Summary of the weight data in a) for day 7 which also includes the NIP-HA control. A significant difference in weight was observed between mice vaccinated with Xcl11-HA and the non targeted controls NIP-HA and C11A-HA. c) Titration of Xcl1-HA DNA used to immunize mice, before challenge with 5× lethal dose of PR8 14 days post immunization. The numbers in brackets indicate that total amount of DNA used to immunize the mice. d) Mice were challenged with PR8 virus 26 weeks post immunization, and monitored for weight loss until day 9 post challenge.

FIG. 7 provides Table 1.

FIG. 8 is graph comparing expression and secretion of murine and human Xcl1 and human Xcl2 vaccibodies.

FIGS. 9a and 9b are a graph comparing immune response after immunization with Xcl1 and Xcl2 vaccibodies.

FIGS. 10a and 10b are graphs comparing weight loss after challenge with influenza virus 14 days after immunization with Xcl1 and Xcl2 vaccibodies.

### DEFINITIONS

As used herein, the term “immune response” refers to a response by the immune system of a subject. For example, immune responses include, but are not limited to, a detectable alteration (e.g., increase) in Toll receptor activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade) cell-mediated immune responses (e.g., responses mediated by T cells (e.g., antigen-specific T cells) and non-specific cells of the immune. system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

As used herein, the term “immunity” refers to protection from disease (e.g., preventing or attenuating (e.g., suppression) of a sign, symptom or condition of the disease) upon exposure to a microorganism (e.g., pathogen) capable of causing the disease. Immunity can be innate (e.g., non-adaptive (e.g., non-acquired) immune responses that exist in the absence of a previous exposure to an antigen) and/or acquired (e.g., immune responses that are mediated by B and T cells following a previous exposure to antigen (e.g., that exhibit increased specificity and reactivity to the antigen)).

As used herein, the term “immunogen” refers to an agent (e.g., a microorganism (e.g., bacterium, virus or fungus) and/or portion or component thereof (e.g., a protein antigen)) that is capable of eliciting an immune response in a subject. In some embodiments, immunogens elicit immunity against the immunogen (e.g., microorganism (e.g., pathogen or a pathogen product)).

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense. In one sense it can refer to a tissue sample. In another sense, it is meant to include a specimen or culture obtained from any source, as well as biological. Biological samples may be obtained from animals (including humans) and encompass fluids, solids, tissues, and gases. Biological samples include, but are not limited to blood products, such as plasma, serum and the like. These examples are not to be construed as limiting the sample types applicable to the present invention. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

As used herein, the term “peptide” refers to a polymer of two or more amino acids joined via peptide bonds or modified peptide bonds. As used herein, the term “dipeptides” refers to a polymer of two amino acids joined via a peptide or modified peptide bond.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified”, “mutant”, and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 20 amino acids long, usually at least 50 amino acids long or longer, and span the portion of the polypeptide required for intermolecular binding of the compositions with its various ligands and/or substrates.

As used herein, the term “purified” or “to purify” refers to the removal of contaminants from a sample. For example, antigens are purified by removal of contaminating proteins. The removal of contaminants results in an increase in the percent of antigen (e.g., antigen of the present invention) in the sample.

The term “variant” may be used interchangeably with the term “mutant.” Variants include insertions, substitutions, transversions, truncations, and/or inversions at one or more locations in the amino acid or nucleotide sequence, respectively. The phrases “variant polypeptide”, “polypeptide”, “variant” and “variant enzyme” mean a polypeptide/protein that has an amino acid sequence that has been modified from the amino acid sequence of native Xcl1. The variant polypeptides include a polypeptide having a certain percent, e.g., 80%, 85%, 90%, 91%, 92%, 93%, 94%, 95%, 96%, 97%, 98%, or 99%, of sequence identity with Xcl1.

“Variant nucleic acids” can include sequences that are complementary to sequences that are capable of hybridizing to the nucleotide sequences presented herein. For example, a variant sequence is complementary to sequences capable of hybridizing under stringent conditions, e.g., 50° C. and 0.2×SSC (1×SSC=0.15 M NaCl, 0.015 M sodium citrate, pH 7.0), to the nucleotide sequences presented herein. More particularly, the term variant encompasses sequences that are complementary to sequences that are capable of hybridizing under highly stringent conditions, e.g., 65° C. and 0.1×SSC, to the nucleotide sequences presented herein. The melting point (Tm) of a variant nucleic acid. may be about 1, 2, or 3° C. lower than the Tm of the wild-type nucleic acid. The variant nucleic acids include a. polynucleotide having a certain percent, e.g., 80%, 85%, 90%, 95%, or 99%, of sequence identity with the nucleic acid encoding Xcl1 or encoding the monomeric protein which can form the homodimeric protein according to invention.

The term “homodimeric protein” as used herein refers to a protein comprising two individual identical strands of amino acids, or subunits held together as a single, dimeric protein by either hydrogen bonding, ionic (charged) interactions, actual covalent disulfide bonding, or some combination of these interactions.

The term “dimerization motif”, as used herein, refers to the sequence of amino acids between the antigenic unit and the targeting unit comprising the hinge region and the optional second domain that may contribute to the dimerization. This second domain may be an immunoglobulin domain, and optionally the hinge region and the second domain are connected through a linker. Accordingly the dimerization motif serve to connect the antigenic unit and the targeting unit, but also contain the hinge region that facilitates the dimerization of the two monomeric proteins into a homodimeric protein according to the invention.

The term “targeting unit” as used herein refers to a unit that delivers the protein with its antigen to a target cell (e.g., cross presenting dendritic cells).

The term “hinge region” refers to a peptide sequence of the homodimeric protein that facilitates the dimerization, such as through the formation of an interchain covalent bond(s), e.g. disulfide bridge(s). The hinge region may be Ig derived, such as hinge exons h1+h4 of an Ig, such as IgG3.

### DETAILED DESCRIPTION OF THE INVENTION

The present invention relates to recombinant fusion proteins targeted to dendritic cells and uses thereof. In particular, the present invention relates to fusion proteins (vaccibodies) comprising a targeting component, an antigen, a linker region and an antibody component and uses of such homodimeric fusion proteins, or DNA encoding such fusion proteins, to trigger immune responses.

Dendritic cells (DCs) exert their functions of immune sentinels in different anatomical places. The DCs that reside in the parenchyma of nonlymphoid tissues (NLT) are called interstitial DCs (int-DCs). These DCs shuttle tissue antigens to draining lymph nodes (LNs), where they are called migratory DCs (mig-DCs). In mouse skin, DCs make up epidermal Langerhans cells (LCs) and three major subsets of dermal DCs: CD11bhiCD24 DCs, CD11b-CD24+CD1032 DCs, and CD11bCD24+CD103+ DCs (1), hereafter referred to as CD11b+ DCs, CD103 DCs, and CD103+ DCs (Table I). Although LCs and all dermal DC subsets constitutively migrate from skin to cutaneous LN (CLN), the CD103+ DCs stand out as the most potent subset for presenting keratinocyte-derived Ags to CD8 T cells in the CLN (1). This capacity is reminiscent of the high efficiency of lymphoid tissue (LT)-resident CD8α+ DCs for cross-presentation (1). CD103+ int-DCs are also found in other anatomical places such as lung and gut. The development of CD103+ int-DCs and LT-resident CD8a+DCs selectively depends on a common set of transcription factors (2, 3). Hence, these mouse DC populations may belong to a unique category of CD8a+-type DCs (1).

CD8a+-type DCs exist in human and sheep, where their identification was based on their expression of a unique transcriptional fingerprint shared with mouse spleen CD8α+ DCs (4, 5) and on their efficiency for Ag cross-presentation (5-9). A universal classification of DCs into five major subsets irrespective of tissues and species: monocytederived inflammatory DCs, LCs, plasmacytoid DCs, CD11b+-type DCs, and CD8a+-type DCs has been published (1). The chemokine receptor XCR1 is specifically expressed by CD8a+-type DCs in mouse spleen, human blood, and sheep lymph (4-7, 10). The function of Xcr1 was first unveiled by the group of R. Kroczek (10), who showed that CD8+ T cell cross-priming depends on their ability to secrete the Xcr1 ligand XCL1 in experimental models where either the OVA coupled to an anti-CD205 Ab or OVA-expressing allogeneic pre-B cells are administrated in vivo. Xcr1 expression on CD8α+ DCs was also found critical for the optimal induction of CD8+ T cell responses upon Listeria monocytogenes infection (6).

Examples of fusion protein vaccines include, e.g., WO 2004/076489, US20070298051, EP920522, Fredriksen A B et al. (Mol Ther 2006; 13:776-85) and Fredriksen A B and Bogen B (Blood 2007; 110: 1797-805); each of which is herein incorporated by reference in its entirety. Embodiments of the present invention provide a recombinant fusion protein including the Xcl1 or Xcl2 chemokines that target Xcr1 (e.g., vaccibodies). The vaccibodies of embodiments of the present invention provide the advantage of enhanced immune responses against the antigen, in particular CD8+ T cell responses.

Numerous studies have shown that targeting antigens to antigen presenting cells (APCs) enhance the immune response. However, not all APCs have the ability to induce CD8+ T-cells. Recent publications indicate that Xcr1 is exclusively expressed on cross-presenting DCs which have this ability. While other targeting methods are pursued in order to target antigens to cross-presenting DCs (such as targeting towards DEC205), these receptors, are often expressed on other populations of APCs as well. Targeting via Xcl1 or Xcl2 thus ensures a highly specific targeting approach.

Accordingly, embodiments, of the present invention provide fusion proteins comprising human or mouse Xcl1 or Xcl2 fused to an immunoglobulin and/or an immunogen. Xcl1 and Xcl2 target fusion polypeptides to the receptor Xcr1. Xcl1 is described by Genbank Accession numbers NM_{—}002995 (human nucleic acid) and NM_{—}008510 (mouse nucleic acid). Embodiments of the present invention further utilize variants, homologs and mimetics of Xcl1 (described in more detail below).

Sequences of human and mouse Xcl1 polypeptides are described by VGTEVLEESSCVNLQTQRLPVQKIKTYIIWEGAMRAVIFVTKRGLKICADPEAKWVK AAIKTVDGRASTRKNMAETVPTGAQRSTSTAITLTG (SEQ ID NO:1; mouse) and VGSEVSDKRTCVSLTTQRLPVSRIKTYTITEGSLRAVIFITKRGLKVCADPQATWVRD VVRSMDRKSNTRNNMIQTKPTGTQQSTNTAVTLTG (SEQ ID NO:2; human). The amino acid sequence of human Xcl2 is described by VGSEVSHRRTCVSLTTQRLPVSRIKTYTITEGSLRAVIFITKRGLKVCADPQATW VRDVVRSMDRKSNTRNNMIQTKPTGTQQSTNTAVTLTG (SEQ ID NO: 3; human) and the nucleic acid sequence ATGAGACTTCTCATCCTGGCCCTCCTTGGCATCTGCTCTCTCACTGCATACATTGT GGAAGGTGTAGGGAGTGAAGTCTCACATAGGAGGACCTGTGTGAGCCTCACTAC CCAGCGACTGCCAGTTAGCAGAATCAAGACCTACACCATCACGGAAGGCTCCTT GAGAGCAGTAATTITATIACCAAACGTGGCCTAAAAGTCTGTGCTGATCCACAA GCCACGTGGGTGAGAGACGTGGTCAGGAGCATGGACAGGAAATCCAACACCAG AAATAACATGATCCAGACCAAGCCAACAGGAACCCAGCAATCGACCAATACAGC TGTGACCCTGACTGGCTAG (SEQ ID NO: 4; human). Native Xcl2 is expressed with a 21 amino acid signal sequence: MRLLILALLGICSLTAYIVEG (SEQ ID NO: 5).

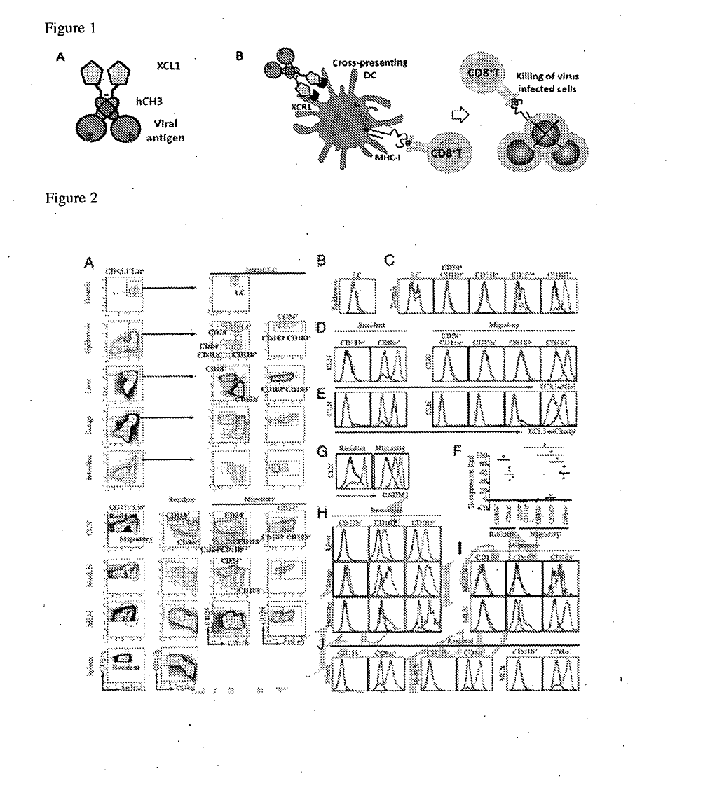

An exemplary fusion protein of embodiments of the present invention is shown in FIG. 1. In some embodiments, the fusion protein comprises Xcl1 as a targeting unit (Xcl2 may be substituted for Xcl1), human IgG3 dimerization domain (hCH3) and an antigenic unit consisting of, but not limited to, an antigen. The dimerization causes the vaccine molecule to be bivalent both in terms of targeting units (Xcl1 or Xcl2) and antigen. The present invention is not limited to a particular mechanism. Indeed, an understanding of the mechanism is not necessary to practice the present invention. Nonetheless, it is contemplated that targeting vaccibodies to Xcr1 on cross-presenting DCs induces presentation of viral peptides on MHC-I to CD8⁺ T-cells. The latter, once activated, can then kill virally infected cells or other targeted cells presenting the same peptide/MHC complex.

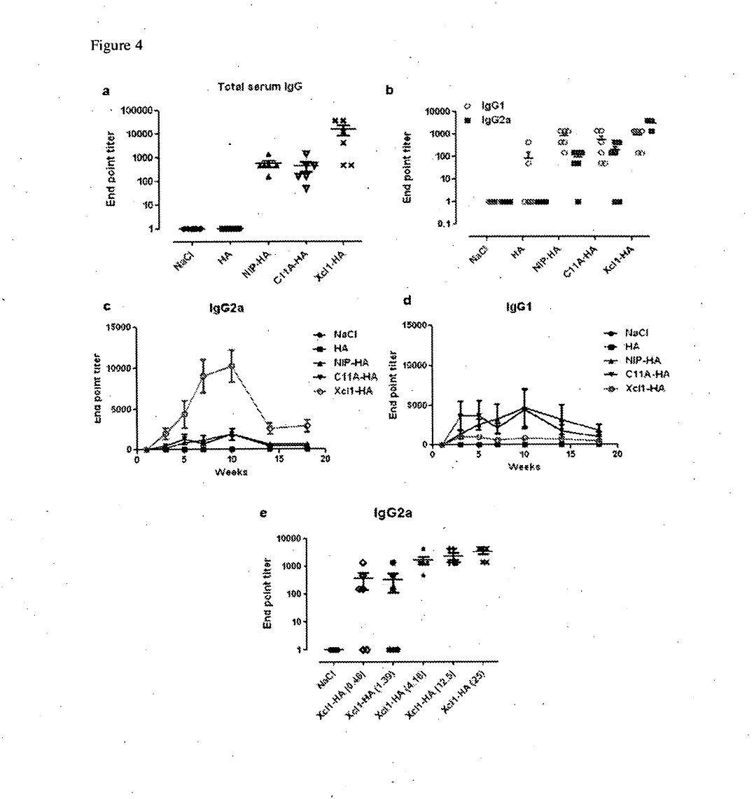

Experiments conducted during the course of development of embodiments of the present invention demonstrated that Xcl11/2-targeted vaccibodies performs better as DNA vaccines when tested in comparison with non targeted vaccibodies vaccibodies using influenza virus hemaglutinin (HA) as an antigenic unit. Xcl1/2-targeted vaccibodies induced stronger IgG2a antibody responses, and better protection in mice against a lethal influenza infection (See e.g., Example 1 and FIGS. 4-6).

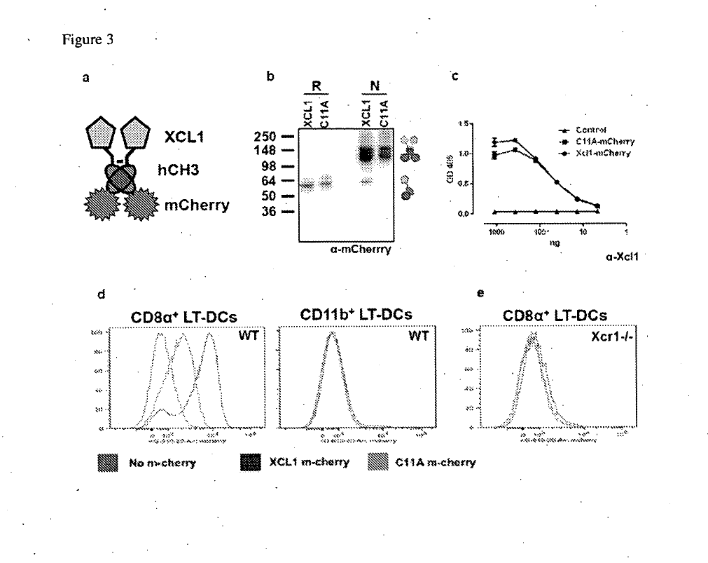

Experiments described herein generated vaccibody constructs which contain Xcl1 as a targeting unit in combination with the fluorescent protein mCherry as well as the viral antigen hemaglutinin (HA) from influenza virus. Initially, the expression and secretion of the Xcl1-mCherry vaccibodies was evaluated. Binding was analysed on DCs enriched from either lymph nodes or skin. Xcl1-mCherry was only observed to bind to the CD8+ residential DCs and the CD 103+ migratory DCs, both of which are known to cross-present antigens on MHC-I (FIGS. 2-3). To evaluate the effect of Xcl1 targeting in a vaccine setting we used the Xcl1-HA vaccibody to vaccinate mice, and subsequently harvest serum samples to evaluate IgG levels. Xcl1-HA induced a strong and lasting IgG2a response. Next, the ability of Xcl1-HA to protect mice from a lethal challenge of influenza virus was evaluated. Mice vaccinated with Xcl1-HA were completely protected from the virus after a single vaccination with 25 μg DNA, and we were able to titrate the amount of DNA used to immunize the mice down to 4.16 μg and still get full protection. This indicates that Xcl1/2-targeting provides a potent method for inducing protective immune responses.

Fusion protein vaccines (e.g., vaccibodies) according to the present invention may be recombinant Ig-based homodimeric vaccines, each chain being composed of a targeting unit directly attached to Ig hinge and CH3, the combination of which induces covalent homodimerization.

Fusion proteins comprising Xcl1/2 polypeptides, including variants thereof, and different antigenic units are preferably constructed and expressed as functional proteins. In particular, the present invention relates to the utilization of Xcl1/2 and their natural isoforms in fusion vaccines to target antigen delivery to antigen-presenting cells. In particularly preferred embodiments, the antigen presenting cell is a cross-presenting dendritic cell or other APC that present Xcr1. Included within the present invention are DNA vaccines encoding a fusion protein that targets antigen delivery to Xcl1/2 receptors (Xcr1) on professional antigen-presenting cells (APC). In preferred embodiments, it is contemplated that targeting vaccibodies to Xcr1 on cross-presenting DCs induces presentation of viral peptides on MHC-I to CD8+ T-cells. The latter, once activated, can then kill virally infected cells presenting the same peptide/MHC complex.

The recombinant proteins according to the present invention may be human antibody-like molecules useful in vaccines, including cancer vaccines. These molecules, also referred to as vaccibodies, bind APC and trigger both T cell and B cell immune responses: Moreover, it is contemplated that vaccibodies bind divalently to APC to promote a more efficient induction of a strong immune response. Vaccibodies preferably comprise a dimer of a monomeric unit that consists of a targeting unit with specificity for a surface molecule on APC, connected through a dimerization motif, such as a hinge region and a Cγ3 domain, to an antigenic unit, the later being in the COOH-terminal or NH₂-terminal end. The present invention also relates to a DNA sequence coding for this recombinant protein, to expression vectors comprising these DNA sequences, cell lines comprising said expression vectors, to treatment of mammals preferentially by immunization by means of vaccibody DNA, vaccibody RNA, or vaccibody protein, and finally to pharmaceuticals and kits comprising such molecules.

The dimerization motif in the proteins according to the present invention may be constructed to include a hinge region and an immunoglobulin domain (e.g. Cγ3 domain), e.g. carboxyterminal C domain (C_{H}3 domain), or a sequence that is substantially homologous to said C domain. The hinge region may be Ig derived and contributes to the dimerization through the formation of an interchain covalent bond(s), e.g. disulfide bridge(s). In addition, it functions as a flexible spacer between the domains allowing the two targeting units to bind simultaneously to two target molecules on APC expressed with variable distances. The immunoglobulin domains contribute to homodimerization through noncovalent interactions, e.g. hydrophobic interactions. In a preferred embodiment the C_{H}3 domain is derived from IgG. These dimerization motifs may be exchanged with other multimerization moieties (e.g. from other Ig isotypes/subclasses). Preferably the dimerization motif is derived from native human proteins, such as human IgG.

It is to be understood that the dimerization motif may have any orientation with respect to antigenic unit and targeting unit. In one embodiment the antigenic unit is in the COOH-terminal end of the dimerization motif with the targeting unit in the N-terminal end of the dimerization motif. In another embodiment the antigenic unit is in the N-terminal end of the dimerization motif with the targeting unit in the COOH-terminal end of the dimerization motif.

International application WO 2004/076489, which is hereby incorporated by reference, discloses nucleic acid sequences and vectors, which may be used according to the present invention.

The proteins according to the present invention may be suitable for induction of an immune response against any polypeptide of any origin. Any antigenic sequence of sufficient length that include a specific epitope may be used as the antigenic unit in the proteins according to the invention. Accordingly in some embodiments, the antigenic unit comprises an amino acid sequence of at least 9 amino acids corresponding to at least about 27 nucleotides in a nucleic acids sequence encoding such antigenic unit. Such an antigenic sequence may be derived from cancer proteins or infectious agents. Examples of such cancer sequences are telomerase, more specifically hTERT, tyrosinase, TRP-1/TRP-2 melanoma antigen, prostate specific antigen and idiotypes. The infectious agents can be of bacterial, e.g. tuberculosis antigens and OMP31 from brucellosis, or viral origin, more specifically HIV derived sequences like e.g. gp120 derived sequences, glycoprotein D from HSV-2, and influenza virus antigens like hemagglutinin, nuceloprotein and M2. Insertion of such sequences in a vaccibody format might also lead to activation of both arms of the immune response. Alternatively the antigenic unit may be antibodies or fragments thereof, such as the C-terminal scFv derived from the monoclonal Ig produced by myeloma or lymphoma cells, also called the myeloma/lymphoma M component in patients with B cell lymphoma or multiple myeloma.

The vaccibody protein, vaccibody DNA, or vaccibody RNA or the present invention may be utilized for immunization of a subject, for example, by intramuscular or intradermal injection with or without a following electroporation.

The targeting unit of the proteins according to the invention targets the protein to APC through binding to chemokine receptors. In particularly preferred embodiments, the chemokine receptor is Xcr1.

The various units of fusion proteins according to the present invention may be operably linked via standard molecular biology methods, and the DNA transfected into a suitable host cell, such as NS0 cells, 293E cells, CHO cells or COS-7 cells. The transfectants produce and secrete the recombinant proteins.

The present invention further relates to a pharmaceutical comprising the above described recombinant based proteins, DNA/RNA sequences, or expression vectors according to the invention. Where appropriate, this pharmaceutical additionally comprises a pharmaceutically compatible carrier. Suitable carriers and the formulation of such pharmaceuticals are known to a person skilled in the art. Suitable carriers are, for example, phosphate-buffered common salt solutions, water, emulsions, e.g. oil/water emulsions, wetting agents, sterile solutions etc. The pharmaceuticals may be administered orally or parenterally. The methods of parenteral administration comprise the topical, intra-arterial, intramuscular, subcutaneous, intramedullary, intrathekal, intraventricular, intravenous, intraperitoneal or intranasal administration. The suitable dose is determined by the attending physician and depends on different factors, e.g. the patient's age, sex and weight, the kind of administration etc. Furthermore, the present invention relates to a vaccine composition against cancer or infectious diseases comprising an immunologically effective amount of the nucleic acid encoding the molecule of the invention or degenerate variants thereof, wherein said composition is able to trigger both a T-cell- and B-cell immune response.

The present invention also relates to a kit comprising vaccibody DNA, RNA, or protein for diagnostic, medical or scientific purposes.

The invention further relates to a method of preparing the recombinant molecule of the invention comprising, transfecting the vector comprising the molecule of the invention into a cell population; culturing the cell population; collecting recombinant protein expressed from the cell population; and purifying the expressed protein.

The above described nucleotide sequences may preferably be inserted into a vector suited for gene therapy, e.g. under the control of a specific promoter, and introduced into the cells. In a preferred embodiment the vector comprising said DNA sequence is a virus, for example, an adenovirus, vaccinia virus or an adeno-associated virus. Retroviruses are particularly preferred. Examples of suitable retroviruses are e.g. MoMuLV or HaMuSV. For the purpose of gene therapy, the DNA/RNA sequences according to the invention can also be transported to the target cells in the form of colloidal dispersions. They comprise e.g. liposomes or lipoplexes.

The present invention also encompasses the use of polypeptides or domains or motifs within the polypeptides having a degree of sequence identity or sequence homology with amino acid sequence(s) defined herein or with a polypeptide having the specific properties defined herein. The present invention encompasses, in particular, peptides having a degree of sequence identity with Xcl1/2, or homologs thereof. Here, the term “homolog” means an entity having sequence identity with the subject amino acid sequences or the subject nucleotide sequences, where the subject amino acid sequence preferably is the amino acid sequence of Xcl1/2.

In one aspect, the homologous amino acid sequence and/or nucleotide sequence should provide and/or encode a polypeptide which retains the functional activity and/or enhances the activity of a Xcl1/2 polypeptide.

In the present context, a homologous sequence is taken to include an amino acid sequence which may be at least 80%, at least 85%, at least 90%, at least 95%, at least 96%, at least 97%, at least 98% or at least 99%, identical to the subject sequence. Typically, the homologs will comprise the same active sites and other functional sequences as the subject amino acid sequence. Although homology can also be considered in terms of similarity (e.g., amino acid residues having similar chemical properties/functions), in the context of the present invention it is preferred to express homology in terms of sequence identity.

Sequence identity comparisons can be conducted by eye, or more usually, with the aid of readily available sequence comparison programs. These commercially available computer programs use complex comparison algorithmuns to align two or more sequences that best reflect the evolutionary events that might have led to the difference(s) between the two or more sequences. Therefore, these algorithms operate with a scoring system rewarding alignment of identical or similar amino acids and penalizing the insertion of gaps, gap extensions and alignment of non-similar amino acids, The scoring system of the comparison algorithms include:
i) assignment of a penalty score each time a gap is inserted (gap penalty score),
ii) assignment of a penalty score each time an existing gap is extended with an extra position (extension penalty score),
iii) assignment of high scores upon alignment of identical amino acids, and
iv) assignment of variable scores upon alignment of non-identical amino acids.
   Most alignment programs allow the gap penalties to be modified. However, it is preferred to use the default values when using such software for sequence comparisons.

The scores given for alignment of non-identical amino acids are assigned according to a scoring matrix also called a substitution matrix. The scores provided in such substitution matrices are reflecting the fact that the likelihood of one amino acid being substituted with another during evolution varies and depends on the physical/chemical nature of the amino acid to be substituted. For example, the likelihood of a polar amino acid being substituted with another polar amino acid is higher compared to being substituted with a hydrophobic amino acid. Therefore, the scoring matrix will assign the highest score for identical amino acids, lower score for non-identical but similar amino acids and even lower score for non-identical non-similar amino acids. The most frequently used scoring matrices are the PAM matrices (Dayhoff et al. (1978), Jones et al. (1992)), the BLOSUM matrices (Henikoff and Henikoff (1992)) and the Gonnet matrix (Gonnet et al. (1992)).

Suitable computer programs for carrying out such an alignment include, but are not limited to, Vector NTI (Invitrogen Corp.) and the ClustalV, ClustalW and ClustalW2 programs (Higgins D G & Sharp P M (1988), Higgins et al. (1992), Thompson et al. (1994), Larkin et al. (2007). A selection of different alignment tools is available from the ExPASy Proteomics server. Another-example of software that can perform sequence alignment is BLAST (Basic Local Alignment Search Tool), which is available from the webpage of National Center for Biotechnology Information (Altschul et al. (1990) J. Mol. Biol. 215; 403-410).

Once the software has produced an alignment, it is possible to calculate % similarity and % sequence identity. The software typically does this as part of the sequence comparison and generates a numerical result.

In one embodiment, it is preferred to use the ClustalW software for performing sequence alignments. Preferably, alignment with ClustalW is performed with the following parameters for pairwise alignment:

| | |
|---|---|
| Substitution matrix: | Gonnet 250 |
| Gap open penalty: | 20 |
| Gap extension penalty: | 0.2 |
| Gap end penalty: | None |

ClustalW2 is for example made available on the internet by the European Bioinformatics Institute at the EMBL-EBI webpage under tools—sequence analysis—ClustalW2.

In another embodiment, it is preferred to use the program Align X in Vector NTI (Invitrogen) for performing sequence alignments. In one embodiment, Exp10 has been may be used with default settings:

Gap opening penalty: 10
Gap extension penalty: 0.05
Gapseparation penalty range: 8
Score matrix: blosum62mt2
Thus, the present invention also encompasses the use of variants, homologues and derivatives of any amino acid sequence of a protein, polypeptide, motif or domain as defined herein, particularly those of Xcl1/2.

The sequences, particularly those of variants, homologues and derivatives of Xcl1/2, may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino-acids with uncharged polar head groups having similar hydrophilicity values include leucine, isoleucine, valine, glycine, alanine, asparagine, glutamine, serine, threonine, phenylalanine, and tyrosine.

The present invention also encompasses conservative substitution (substitution and replacement are both used herein to mean the interchange of an existing amino acid residue, with an alternative residue) that may occur, e.g. like-for-like substitution such as basic for basic, acidic for acidic, polar for polar etc. Non-conservative substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Conservative substitutions that may be made are, for example within the groups of basic amino acids (Arginine, Lysine and Histidine), acidic amino acids (glutamic acid and aspartic acid), aliphatic amino acids (Alanine, Valine, Leucine, Isoleucine), polar amino acids (Glutamine, Asparagine, Serine, Threonine), aromatic amino acids (Phenylalanine, Tryptophan and Tyrosine), hydroxyl amino acids (Serine, Threonine), large amino acids (Phenylalanine and Tryptophan) and small amino acids (Glycine, Alanine).

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I -phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#}*, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-conservative substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al. (1992), Horwell D C. (1995).

In one embodiment, the variant targeting unit used in the homodimeric protein according to the present invention is variant having the sequence of Xcl1/2 and having at least at least 65%, at least 70%, at least 75%, at least 78%, at least 80%, at least 85%, at least 90%, at least 95%, at least 96%, at least 97%, at least 98% or at least 99% amino acid sequence identity therewith.

In one aspect, preferably the protein or sequence used in the present invention is in a purified form. A “variant” or “variants” refers to proteins, polypeptides, units, motifs, domains or nucleic acids.

### EXPERIMENTAL

The following examples are provided in order to demonstrate and further illustrate certain preferred embodiments and aspects of the present invention and are not to be construed as limiting the scope thereof.

### Example 1

### Materials and Methods

### Cell Lines, Virus and Antibodies:

HEK293E cells were used for expression of HA-vaccibodies, and for transfecting Xcr1-eGFP. Antibodies towards Xcl1 was obtained from Lifespan Biosciences (C-16241), while antibodies α-HA (H-36-4-52), α-human IgG3 (HP-6017) and α-mCherry were purified in the lab. For serum immunoglobulin ELISA α-mouse IgG1-bio (BD Pharmingen, clone 10.9), α-mouse IgG2α-bio (BD Pharmingen, clone 8.3), α-mouse IgG2b-bio (BD Pharmingen, clone R12-3). Influenza virus strain A/PR/8/34(H1N1) was obtained from the Norwegian Institute of Public Health.

Purification of Xcl1-mCherry Vaccibodies:

Stable transfectants were generated by electropporating 2×10⁷ NS0 cells in PBS with 40 μg of Xcl1-mCherry or Xcl1(C11A)-mcherry DNA. The cells were transferred to fresh RPMI medium and left to recover in a T-25 flask at 37° C. for 24 hours without selection. Next day, G418 was added to a final concentration of 800 μg/ml and cells seeded in 96-well plates at a density of 5×10⁴ cells pr well. Colonies of stably transfected cells appeared after 2-3 weeks. Stable transfectants were subsequently expanded in rollerbottles, and supernatant collected after 5 days and applied on an α-mCherry column connected to an Äktaprime Plus (GE Healthcare). Bound vaccibodies were washed with PBS, eluted in 0.1 M Glycin-HCl pH 10.5, and immediately dialyzed against PBS.

### ELISA:

96-well ELISA plates (Costar) were coated with 2 μg/ml of inactivated PR8 influenza virus (Supplier), and incubated ON at 4° C. The plates were then incubated with 150 μl/well blocking buffer (1% (w/v) BSA in PBS with 0.02% (w/v) NaAzide) for 1 h at RT. After washing the plates, serum samples were diluted 1:50, and subsequently serial diluted 1:3, in ELISA buffer (0.1% (w/v) BSA in PBS with 0.02% (w/v) NaAzide). ELISA plates were incubated with serum samples overnight at 4° C. Next, plates were washed and incubated with 1 μg/ml biotinylated antibodies specific for IgG1, IgG2a, IgG2b or IgG3 (BP Pharmingen), and incubated for 1 h 37° C. After washing, the plates were incubated with streptavidin-ALP (GE Healthcare (RPN1234V), 1:3000) for 45 min at RT. ELISAs were developed by adding 100 μl/well of substrate buffer (1 μg/ml phosphate substrate (Sigma, P4744), After 30 min OD₄₀₅ was measured on a Tecan Sunrise. For analyzing total serum immunoglobulin, plates were incubated with ALP conjugated anti-mouse Fc (sigma) (1:300): Antibody titer was determined as the highest dilution of a serum sample with OD values > (mean+5×SD) of NaCl vaccinated mice.

### Mice

Xcr1tm1Dgen mice (Xcr1-bGal) (6, 10) generated by Deltagen were bred in Centre d'Immunologie Maiseille-Luminy animal care facilities. C57BU6J Q:8 mice were purchased from Charles River Laboratories (France). Studies were performed in accordance with institutional regulations governing animal care and use.

### DC Isolation and Sorting Strategy

DCs were isolated from various organs by a combination of enzymatic digestion, mechanical disruption, and gradient density enrichment (11). Sorting of CLN DCs was performed as described previously (12).

### Abs and Flow Cytometry

Most Abs were purchased from eBioscience or BD Biosciences. Identification of mig-DCs was based on their specific pattern of expression of CD 11c and MHC class II. CADM1 staining was performed with a chicken anti-SynCAM/TSLC1 Ab (clone 3.E.1) revealed with a goat antichicken IgG. XCR1 expression was detected using fluorescein di-b-D-galactopyranoside as a fluorogenic substrate for b-galactosidase (bGal) (6). In CLNs, XCR1 expression was also detected by using recombinant mouse XCL1 covalently coupled to the red fluorescent protein mCherry.

### Results

High level of XCR1 expression is selective for CD103+ int-DCs in skin and CD103+ mig-DCs in CLNs

To investigate which DC subsets express XCR1 in skin and CLNs, we exploited a reporter mutant mouse model expressing b-galactosidase (bGal) in place of XCR1. In skin, the int-DC subsets encompass the epidermal LCs and the dermal subsets CD103+ DCs, CD1032 DCs, CD11b+ DCs, and CD11bCD24 TI DCs (Table I). In skin int-DCs, bGal activity was high in CD103+ DCs, low in CD103 DCs, and undetectable in CD11bCD24 DCs, CD11b DCs, and LCs (FIG. 2A, 2B). In CLNs, only LTresident CD8α+ DCs and CD 103+ mig-DCs expressed XCR1 (FIG. 2C, 2E). The use of fluorescently labeled recombinant mouse XCL1 to stain CLN cells gave a strong and highly specific signal on LT-resident CD8α+ DCs and CD103+ mig-DCs from wild-type mice (FIG. 2D). These data confirmed that the protein XCR1 is specifically expressed on LT-resident CD8α+ DCs and CD103+ mig-DCs and validated the use of bGal activity as a faithful reporter of XCR1 expression. Therefore, in skin and CLNs, a high level of XCR1 expression is selective for CD8a+-type DCs.

XCR1 expression defines CD8a+-type DCs in visceral organs and their draining LNs

We next analyzed XCR1 expression on DCs residing in different tissues. As in skin and CLNs, three main populations were defined in the liver, lungs, and the small intestine: CD11b+DCs, CD103+ DCs, and CD103 DCs (Table I). In these organs, XCR1 expression was high in CD103+ int-DCs, intermediate in CD103 int-DCs, and not detected in CD11b+ int-DCs (FIG. 2G), as observed in the skin. In the mig-DCs from mesenteric LNs (MLNs) and mediastinal LNs (MedLNs) draining, respectively, the intestine and the lung, XCR1 expression remained highest in the CD103+ subset (FIG. 2H). Despite the use of an inhibitor of endogeneous Gal activity, the CD103+ int-DCs in the intestine and the CD103+ mig-DCs in MedLN showed substantial levels of bGal activity in wild-type mice. However, a clear increase over that background signal could be detected in the corresponding subsets isolated from XCR1-bGal mice. Within LT-resident DCs, XCR1 expression remained confined to the CD8a+ subset (FIG. 2I).

In order to generate vaccibodies that target Xcr1 expressing DCs we replaced the endogenous signaling peptide of Xcl1 by that of human IgG3, which was originally included in the vaccibody genetic construct. As a model antigen we used mCherry which can be detected by its intrinsic ability to fluoresce, as well as via specific antibodies generated in our lab (FIG. 3a). In addition to Xcl1-mCherry, we generated a mutated version of Xcl1 where the Cys11 was mutated to an alanine C11A-mCherry). Since Cys11, in addition to Cys48, is involved in forming the only cysteine bridge in Xcl1 it was contemplated that the mutant would be devoid of function². The two vaccibodies were purified on an α-mCherry column, and evaluated for size and dimerization by SDS-PAGE. In the presence of β-mercaptoethanol the two vaccibodies had a size of ˜60 kd, while under non-reducing conditions the size was ˜148 kd indicated that the purified vaccibodies mainly consist of dimers (FIG. 3b). Due to the relative small size of the targeting unit we also confirmed that Xcl1 and C11A were present in the vaccibodies in an ELISA assay where α-Xcl1 was used as the primary antibody (FIG. 3c).

In order to evaluate if Xcl1-targeted vaccibodies bound to the CD8α+ DC population known to express Xcr1, we isolated DCs from spleen and incubated them with Xcl1-mCherry or C11A-mCherry (FIG. 3d): Xcl1-mCherry only bound to CD8+DC and not CD11b+DCs, indicating that the vaccibodies specifically target Xcr1+DCs. To ensure that the binding was specific for Xcr1, DCs were isolated from Xcr1−/− mice and stained with Xcl1- and C11A-mCherry (FIG. 3e). No binding was observed to any of the DC populations in Xcr1−/− mice indicating that the binding to CD8α+ DCs is mediated by Xcr1. Some degree of binding, although significantly less than with Xcl1-mCherry, was also observed with the C11A-mCherry vaccibody, indicating that mutating cystein 11 to alanine does not completely abrogate binding. Since the C11A-mCherry binding was lost in Xcr1−/− mice, it seems to be specific for Xcr1 and not just unspecific background.

Next, Balb/c mice were immunized with 25 μg of DNA encoding the Xcl1-HA vaccibody. As additional controls we included one group of mice immunized with a plasmid expressing PR8 HA alone, and one group immunized with 0.9% NaCl, one group immunized with NIP-HA (scFv specific for the hapten NIP), in addition to C11A-HA. Serum samples were collected 14 days post immunization and initially analyzed for HA specific serum IgG (FIG. 4a). The strongest induction of IgG was observed in mice immunized with Xcl1-HA. Interestingly, when analyzing IgG subclass responses we observed that Xcl1-HA mainly induced IgG2a, and that these levels were significantly higher than any of the other groups (FIG. 4b). This is in contrast to the levels of IgG1 which were comparable in mice immunized with both targeted and non-targeted vaccobodies (FIG. 4b). When monitoring the humoral response over time we observed that the IgG2a titer peaked around 7-10 weeks post immunization, and subsequently declined until reaching a stable level after about 15 weeks (FIG. 4c). For IgG1, an increase in titer was observed with the non-targeted vaccibodies (NIP-HA and C11A-HA), until 10 weeks post immunization (FIG. 4d). This is in contrast to Xcl1-HA vaccibodies where no increase in IgG1 titer was observed after the third week.

To evaluate the potency of targeting antigens to Xcr1 expressing cells we immunized Balb/c mice with decreasing amounts of Xcl1-HA DNA. Serum samples were taken 2 weeks post immunization and analyzed for total IgG, as well as IgG1 and IgG2a. While only a minimal IgG2a response could be observed in mice immunized with 0.46 and 1.39 μg of DNA, moderate levels of IgG2a were observed in serum of mice which received 4.16 μg of DNA (FIG. 4e).

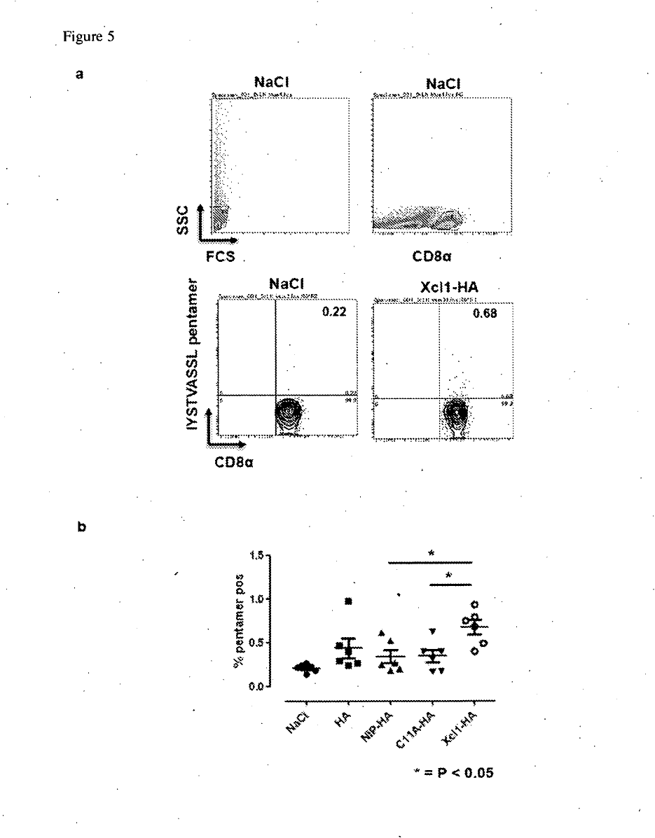

It was contemplated that targeting antigens to cross-priming Xcr1+DC subsets would induce CD8+ T-cells. To test this, we immunized mice with Xcl1-HA and harvested draining lymph nodes (inguinal and auxiliary) 14 days post immunization and stained the isolated cells for HA-specific CD8+ T-cells using the IYSTVASSL pentamer (FIG. 5a). When comparing the percentage of pentamer positive CD8+ T-cells obtained with Xcl1-HA vaccibodies to those obtained with non-targeted controls (NIP-HA or C11A-HA) we observed a significant higher numbers of HA-specific CD8+t-cells with the Xcl1-targeted vaccibodies (Mann-Whitney) (FIG. 5b).

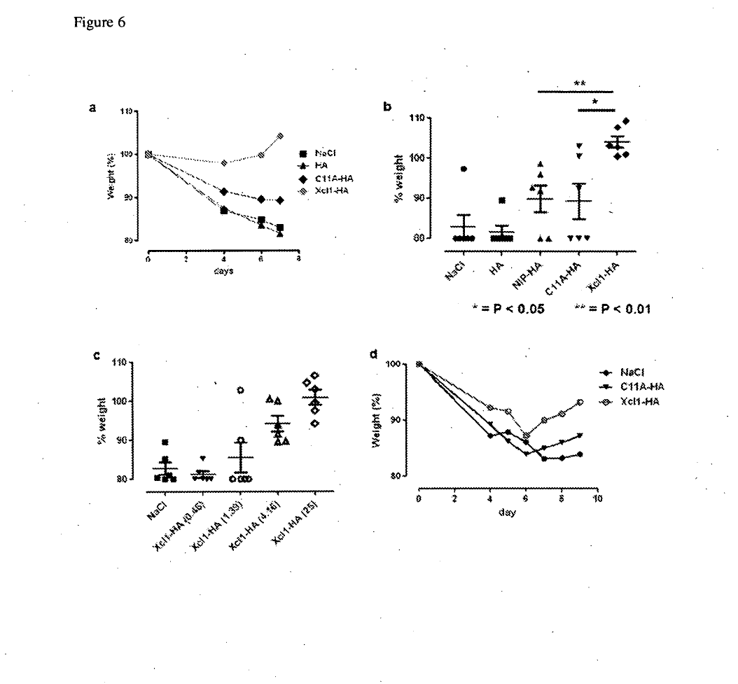

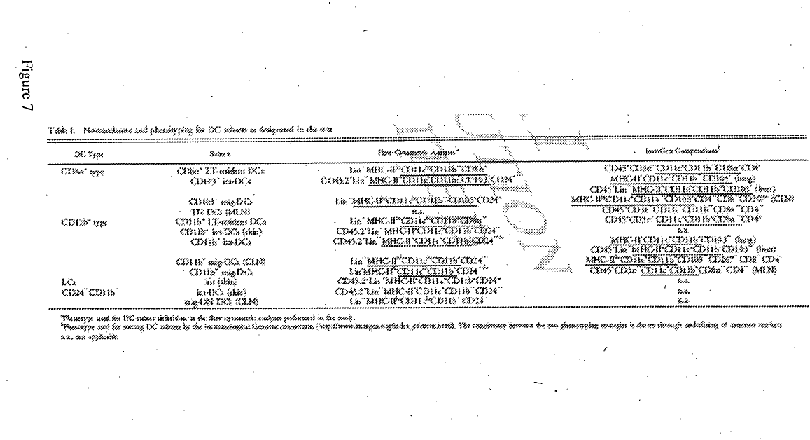

Next we wished to evaluate if the immune response induced by targeting antigen to Xcr1 expressing DCs was sufficient to protect mice against an influenza infection. Balb/c mice were immunized with 25 μg of DNA and challenge with 5× lethal dose of Influenza A/PR/8/34(H1N1) 14 days post immunization. Weight loss was monitored as a sign of disease progression. Mice vaccinated with Xcl1-HA initially lost some weight, but recovered after day 4 post challenge (FIG. 6a). Mice vaccinated with HA alone did not induce a protective response and continued to lose weight until the experiment was terminated on day 7. When evaluating the data for day 7 post challenge for each individual mouse, and including all controls, we observed a significant difference in weight when comparing non-targeted vaccines (HA, NIP-HA and C11A-HA) to Xcl1-HA (Mann-Whitney)(FIG. 6b).

One potential hurdle that arises when trying to transfer DNA vaccination technology into larger animals is that the amount of DNA needed to induce protection increases with the size of the animal. It is therefore preferred that the vaccines are able to induce protection at relative low concentrations. To evaluate the potency of targeting vaccibodies to Xcr1 expressing CD8α+ DCs we titrated the amount of DNA used to vaccinate the mice before challenged with PR8 two weeks post immunization. Mice were consistently protected after immunization with a total of 4.16 μg of Xcl1-HA DNA (FIG. 6c). This correlates well with the amount of DNA needed to induce a consistent immuneresponse as determined by serum IgG titers (FIG. 6c).

To determine if Xcl1-HA vaccibodies had the ability to induce long term protection, we challenged mice with PR8, 26 weeks post immunization and monitored for weight loss (FIG. 6d). Mice immunized with Xcl1-HA initially lost weight, but all but 1 of 5 mice started recovering from the infection after day 6. In contrast mice immunized with NaCl continued to lose weight and by day 9 4/6 mice had to be euthanized. Mice immunized with the mutated C11A-HA, generally lost more weight, but also started to recover after day 6 post infection.

### Example 2

Vaccibodies were produced as described above, except that Xcl2 was substituted for Xcl1 as the targeting unit.

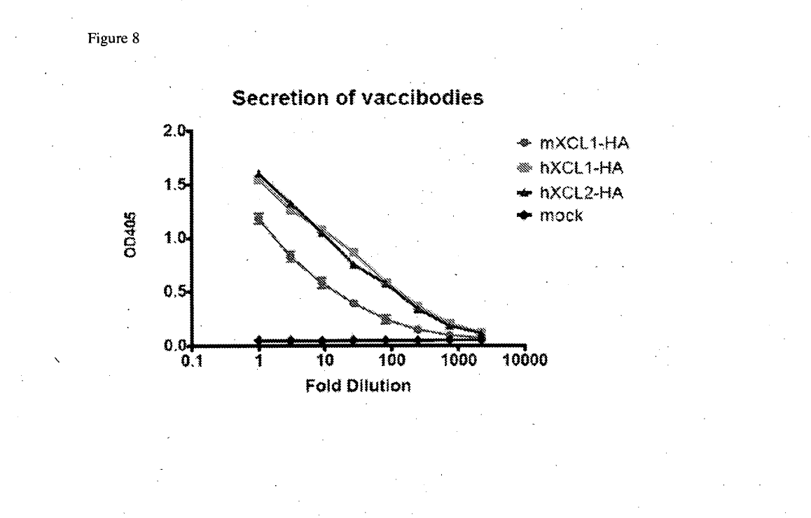

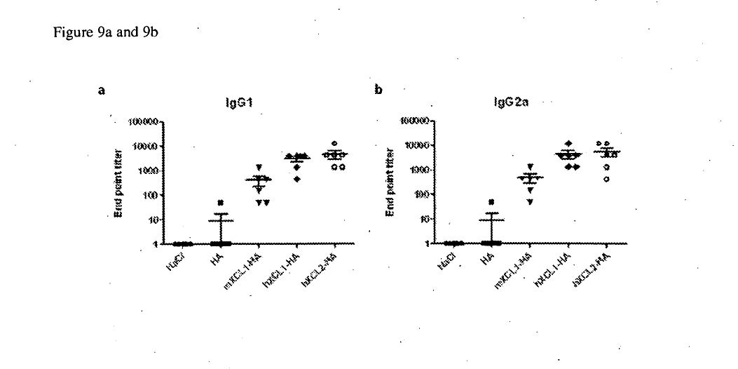

HEK293E cells were transiently transfected with plasmids encoding murine Xcl1-HA (mXcl1), human Xcl1-HA (hXcl1) or human Xcl2-HA (hXcl2) vaccibodies. Supernatants were harvested after 48 h and analyzed for secretion of vaccibodies by ELISA. All three vaccibodies were efficiently expressed and secreted, with hXcl1 and hXcl2 giving better expression than mXcl1. The results are presented in FIG. 8. Next, Balb/c mice were immunized with 25 μg of DNA encoding mXcl1, hXcl1 or hXcl2-HA vaccibodies. Fourteen days post immunization blood samples were collected and serum titers of IgG1 and IgG2a determined by ELISA. Both hXcl1 and hXcl2 induces higher IgG1 and IgG2a responses than mXcl1. The results are presented in FIGS. 9a and 9b. Balb/c mice were then immunized with 25 μg of DNA encoding mXcl1, hXcl1 or hXcl2-HA vaccibodies, and challenged with a lethal dose of influenza virus 14

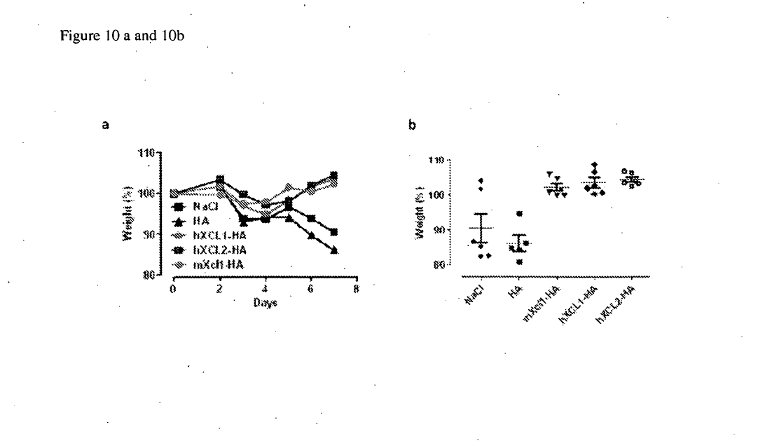

days post vaccination. The results are presented in FIG. 10(a) which shows weight loss monitored for 7 days and used as an indication of disease progression. Mice vaccinated with NaCl or HA alone succumbed to the viral infection while mice vaccinated with mXcl1, hXcl1 or hXcl2 survived the challenge. FIG. 10(b) shows weight loss for all mice on day 7 after infection.

### REFERENCES

1. Guilliams, M., S. Henri, S. Tamoutounour, L. Ardouin, I. Schwartz-Cornil, M. Dalod, and B. Malissen. 2010. From skin dendritic cells to a simplified classification of human and mouse dendritic cell subsets. Eur. J. Immunol. 40: 2089-2094.
2. Ginhoux, F., K. Liu, J. Helft, M. Bogunovic, M. Greter, D. Hashimoto, J. Price, N. Yin, J. Bromberg, S. A. Lira, et al. 2009. The origin and development of nonlymphoid tissue CD103+ DCs. J. Exp. Med. 206: 3115-3130.
3. Edelson, B. T., W: Kc, R. Juang, M. Kohyama, L. A. Benoit, P. A. Klekotka, C. Moon, J. C. Albring, W. Ise, D. G. Michael, et al. 2010. Peripheral CD103+ dendritic cells form a unified subset developmentally related to CD8a+ conventional dendritic cells. J. Exp. Med. 207: 823-836.
4. Robbins, S. H., T. Walzer, D. Dembe'le', C. Thibault, A. Defays, G. Bessou, H. Xu, E. Vivier, M. Sellars, P. Pierre, et al. 2008. Novel insights into the relationships between dendritic cell subsets in human and mouse revealed by genome-wide expression profiling. Genome Biol. 9: R17.
5. Contreras, V., C. Urien, R. Guiton, Y. Alexandre, T. P. Vu Manh, T. Andrieu, K. Crozat, L. Jouneau, N. Bertho, M. Epardaud, et al. 2010. Existence of CD8alike dendritic cells with a conserved functional specialization and a common molecular signature in distant mammalian species. J. Immunol. 185: 3313-3325.
6. Crozat, K., R. Guiton, V. Contreras, V. Feuillet, C. A. Dutertre, E. Ventre, T. P. Vu Manh, T. Baranek, A. K. Storset, J. Marvel, et al. 2010. The XC chemokine receptor 1 is a conserved selective marker of mammalian cells homologous to mouse CD8a+ dendritic cells. J. Exp. Med. 207: 1283-1292.
7. Bachem, A., S. Gu\ddot{}ttler, E. Hartung, F. Ebstein, M. Schaefer, A. Tannert, A. Salama, K. Movassaghi, C. Opitz, H. W. Mages, et al. 2010. Superior antigen crosspresentation and XCR1 expression define human CD11c+CD141+ cells as homologues of mouse CD8+ dendritic cells. J. Exp. Med. 207: 1273-1281.
8. Poulin, L. F., M. Salio, E. Griessinger, F. Anjos-Afonso, L. Craciun, J. L. Chen, A. M. Keller, O. Joffre, S. Zelenay, E. Nye, et al. 2010. Characterization of human DNGR-1+BDCA3+ leukocytes as putative equivalents of mouse CD8a+ dendritic cells. J. Exp. Med. 207: 1261-1271.
9. Jongbloed, S. L., A. J. Kassianos, K. J. McDonald, G. J. Clark, X. Ju, C. E. Angel, C. J. Chen, P. R. Dunbar, R. B. Wadley, V. Jeet, et al. 2010. Human CD141+(BDCA-3)+ dendritic cells (DCs) represent a unique myeloid DC subset that cross-presents necrotic cell antigens. J. Exp. Med. 207: 1247-1260.
10. Dorner, B. G., M. B. Dorner, X. Zhou, C. Opitz, A. Mora, S. G\ddot{}ttler, A. Hutloff, H. W. Mages, K. Ranke, M. Schaefer, et al. 2009. Selective expression of the chemokine receptor XCR1 on cross-presenting dendritic cells determines cooperation with CD8+ T cells. Immunity 31: 823-833.
11. Guilliams, M., K. Crozat, S. Henri, S. Tamoutounour, P. Grenot, E. Devilard, B. de Bovis, L. Alexopoulou, M. Dalod, and B. Malissen. 2010. Skin-draining lymph nodes contain dermis-derived CD103—dendritic cells that constitutively produce retinoic acid and induce Foxp3+ regulatory T cells. Blood 115: 1958-1968.
12. Henri, S., L. F. Poulin, S. Tamoutounour, L. Ardouin, M. Guilliams, B. de Bovis, E. Devilard, C. Viret, H. Azukizawa, A. Kissenpfennig, and B. Malissen. 2010. CD207+CD 103+ dermal dendritic cells cross-present keratinocyte-derived antigens irrespective of the presence of Langerhans cells. J. Exp. Med. 207: 189-206.
13. Subramanian, A., H. Kuehn, J. Gould, P. Tamayo, and J. P. Mesirov. 2007. GSEAP: a desktop application for Gene Set Enrichment Analysis. Bioinformatics 23: 3251-3253.
14. Sathe, P., J. Pooley, D. Vremec, J. Mintern, J. O. Jin, L. Wu, J. Y. Kwak, J. A. Villadangos, and K. Shortman. 2011. The acquisition of antigen crosspresentation function by newly formed dendritic cells. J. Immunol. 186: 5184-5192.
15. Sancho, D., O. P. Joffre, A. M. Keller, N. C. Rogers, D. Marti´nez, P. Hernanz-Falco´n, I. Rosewell, and C. Reis e Sousa. 2009. Identification of a dendritic cell receptor that couples sensing of necrosis to immunity. Nature 458: 899-903.
16. Galibert, L., G. S. Diemer, Z. Liu, R. S. Johnson, J. L. Smith, T. Walzer, M. R. Comeau, C. T. Rauch, M. F. Wolfson, R. A. Sorensen, et al. 2005. Nectinlike protein 2 defines a subset of T-cell zone dendritic cells and is a ligand for class-Irestricted T-cell-associated molecule. J. Biol. Chem. 280: 21955-21964.
17. Zou, L., J. Zhou, J. Zhang, J. Li, N. Liu, L. Chai, N. Li, T. Liu, L. Li, Z. Xie, et al. 2009. The GTPase Rab3b/3c-positive recycling vesicles are involved in crosspresentation in dendritic cells. Proc. Natl. Acad. Sci. USA 106: 15801-15806.
18. Heng, T. S., M. W. Painter, and Immunological Genome Project Consortium. 2008. The Immunological Genome Project: networks of gene expression in immune cells. Nat. Immunol. 9: 1091-1094.
19. Ziegler-Heitbrock, L., P. Ancuta, S. Crowe, M. Dalod, V. Grau, D. N. Hart, P. J. Leenen, Y. J. Liu, G. MacPherson, G. J. Randolph, et al. 2010. Nomenclature of monocytes and dendritic cells in blood. Blood 116: e74-e80.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

1. A DNA vaccine comprising a pharmaceutically acceptable carrier and a nucleic acid encoding a fusion polypeptide comprising a targeting unit comprising an amino acid sequence having at least 80% sequence identity to SEQ ID NOs 1, 2 or 3 and an antigenic unit.

2. The DNA vaccine of claim 1, wherein said targeting unit and said antigenic unit are connected through a dimerization motif.

3. The DNA vaccine of claim 1, wherein said antigenic unit is selected from the group consisting of an antigenic scFv, a bacterial antigen, a viral antigen and a cancer associated or a cancer specific antigen.

4. The DNA vaccine of claim 1, wherein said targeting unit comprises an amino acid sequence having at least 90% sequence identity to SEQ ID NOs 1, 2 or 3.

5. The DNA vaccine of claim 1, wherein said targeting unit comprises an amino acid sequence having at least 95% sequence identity to SEQ ID NOs 1, 2 or 3.

6. The DNA vaccine of claim 1, wherein said targeting unit comprises the amino acid sequence of SEQ ID NOs 1, 2 or 3.

7. The DNA vaccine of claim 1, wherein said polypeptide is present as a dimer after expression.

8. The DNA vaccine of claim 1, wherein said dimerization domain comprises human IgG3 dimerization domain (hCH3).

9. The DNA vaccine of claim 1, wherein said fusion polypeptide binds to Xcr1 on cross-presenting DCs.

10-12. (canceled)

13. A method of inducing an immune response, comprising administering the DNA vaccine of claim 1 to a subject under conditions such that said subject generates an immune response to said antigenic unit.

14. (canceled)


SEQUENCE LISTING (5 sequences)

SEQ ID NO: 1 - protein, 93 aa, Mus musculus
VGTEVLEESSCVNLQTQRLPVQKIKTYIIWEGAMRAVIFVTKRGLKICADPEAKWVKAAIKTVDGRASTRKNMAETVPTGAQRSTSTAITLTG

SEQ ID NO: 2 - protein, 93 aa, Homo sapiens
VGSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 3 - protein, 93 aa, Homo sapiens
VGSEVSHRRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 4 - DNA, 345 bp, Homo sapiens
atgagacttctcatcctggccctccttggcatctgctctctcactgcatacattgtggaaggtgtagggagtgaagtctcacataggaggacctgtgtgagcctcactacccagcgactgccagttagcagaatcaagacctacaccatcacggaaggctccttgagagcagtaatttttattaccaaacgtggcctaaaagtctgtgctgatccacaagccacgtgggtgagagacgtggtcaggagcatggacaggaaatccaacaccagaaataacatgatccagaccaagccaacaggaacccagcaatcgaccaatacagctgtgaccctgactggctag

SEQ ID NO: 5 - protein, 21 aa, Artificial Sequence
  Synthetic
MRLLILALLGICSLTAYIVEG